(19) United States
(12) Patent Application Publication
(10) Pub. No.: US 2004/0122267 A1
(43) Pub. Date: Jun. 24, 2004

(54) Integrated gas to olefins process with recovery and conversion of by-products

(76) Inventors: Jaimes Sher, Houston, TX (US); James Andrew Zboray, Voorhees, NJ (US)

Correspondence Address: EXXONMOBIL CHEMICAL COMPANY, P O BOX 2149, BAYTOWN, TX, 77522-2149, US

(21) Appl. No.: 10/327,767

(22) Filed: Dec. 23, 2002

(51) Int. Cl. C07C 1/00; C07C 2/00

(52) U.S. Cl. 585/324; 585/327; 585/326; 585/329

(57) ABSTRACT

This invention provides a method for producing a light olefin product from a hydrocarbon feed, as well as a method for converting by-product to light olefin. In one embodiment, the process includes production of a synthesis gas from the hydrocarbon feed, conversion of the synthesis gas to an oxygenate composition, and conversion of the oxygenate components to an olefin stream. A by-product stream can also be separated and ultimately converted to additional light olefin product.

### FIELD OF INVENTION

This invention is to a process of producing a light olefin product from a hydrocarbon feed. Specifically, the process is to producing a light olefin product, with recovery of undesirable by-product, and conversion of the by-product to additional light olefin.

### BACKGROUND OF THE INVENTION

Light olefins, defined herein as ethylene, propylene, butylene and mixtures thereof, serve as feeds for the production of numerous important chemicals and polymers. Typically, light olefins are produced by cracking petroleum feeds. Because of the limited supply of competitive petroleum feeds, the opportunities to produce low cost light olefins from petroleum feeds are limited. Efforts to develop light olefin production technologies based on alternative feeds have increased.

An important type of alternate feed for the production of light olefins is oxygenates, such as, for example, alcohols (e.g., methanol and ethanol), dimethyl ether, methyl ethyl ether, diethyl ether, dimethyl carbonate, and methyl formate. Many of these oxygenates may be produced by fermentation, or from synthesis gas derived from natural gas, petroleum liquids, carbonaceous materials, including coal, recycled plastics, municipal wastes, or any organic material. Because of the wide variety of sources, alcohol, alcohol derivatives, and other oxygenates have promise as an economical, non-petroleum source for light olefin production.

U.S. Pat. No. 5,714,662 to Vora et al. discloses one example of producing a light olefin product from a hydrocarbon gas feed. Specifically, this process discloses reforming the hydrocarbon gas feed to produce a synthesis gas, producing an oxygenate stream from the synthesis gas, and converting the oxygenates to produce the light olefin product. The process further includes recycling by-product water produced during the oxygenate conversion to provide various benefits of increased efficiency.

There are, however, concerns common to management of processing plants, which include efficiency of production and purity of the product. Recycle and treatment of by-products and/or unreacted reactants is one manner of addressing such concerns.

U.S. Pat. No. 6,002,019 to Tamhankar et al. discloses a process for producing a petrochemicals product by partial oxidation of a hydrocarbon feed, which includes recycle of by-products. Upon removal of the product from the product gas stream, part or all of the remaining gas stream is passed through a bed of hydrophobic adsorbent. The adsorbent adsorbs the unreacted hydrocarbon from the gas stream. The adsorbed hydrocarbon is purged from the bed with air, and the air-hydrocarbon mixture is recycled to the partial oxidation reactor at the beginning of the process.

Another such example of using a recycle process to enhance productivity is disclosed in U.S. Pat. No. 5,502,243 to Waller et al. This patent discloses a method for oxygenation of acetyl compounds from a hydrocarbon feed stream. The hydrocarbon feed stream is fed with a steam stream and an oxygen stream into a partial oxidation reactor to produce a synthesis gas stream, which then passes into a liquid phase dimethyl ether (DME) reactor system. The product stream comprises DME, methanol, unreacted synthesis gas, and water. Upon separation, the unreacted synthesis gas is recycled to the partial oxidation reactor at the beginning of the process.

A method of producing a light olefin product from a hydrocarbon feed that maximizes the efficiency of production and the purity of the product would be of great advantage. There is also a desire to minimize disposal and/or storage of potentially undesirable or hazardous by-products.

### SUMMARY OF THE INVENTION

This invention provides a method for producing a light olefin product from a hydrocarbon feed, which minimizes by-product formation. The by-production is minimized in that the by-product is recovered and converted to more desirable olefin product(s).

In one embodiment, there is provided a process for producing olefin(s). The process comprises separating an olefin stream into component streams. One component streams is comprised of a light olefin stream and another component stream is comprised of a by-product stream. The light olefin stream is comprised of at least 50 wt % of at least one C₂ to C₄ olefin, based on total weight of the light olefin stream, and the by-product stream comprised of at least 30 wt % hydrocarbons having a boiling point greater than that of propylene, based on total weight of the by-product stream. The by-product stream is passed to a synthesis gas production zone to produce a synthesis gas stream. Preferably, the synthesis gas stream has a molar ratio of hydrogen to carbon oxide of from 1:1 to 5:1.

In another embodiment, the synthesis gas production zone is a partial oxidation process or a reforming process. Preferably, the synthesis gas production zone includes a catalyst. More preferably, the catalyst is a nickel containing catalyst.

In one embodiment, there is included a step of passing the synthesis gas stream to a carbon oxide conversion zone to produce a methanol stream. Desirably, the carbon oxide conversion zone includes a catalyst comprising at least one transition element particularly selected from the group consisting of Ni, Co, Pd, Ru, Rh, Ir, Pt, Os and Fe.

In another embodiment, there is included a step of passing the methanol stream to an oxygenate conversion zone to produce an olefin stream. Preferably, the oxygenate conversion zone includes a molecular sieve catalyst, particularly a catalyst containing a silicoaluminophosphate molecular sieve. More preferably, the silicoaluminophosphate molecular sieve is selected from the group consisting of SAPO-18, SAPO-34, SAPO-35, SAPO-44, SAPO-47, and a mixture thereof.

In one embodiment, the olefin stream is separated into component streams by distillation. Preferably, the distillation is extractive distillation.

In another embodiment, 90 wt % to 98 wt % of the methanol stream that is passed to the oxygenate conversion zone is converted to produce the olefin stream. Alternatively, 98 wt % to less than 100 wt % of the methanol stream that is passed to the oxygenate conversion zone is converted to produce the olefin stream.

The light olefin stream can be used as a conventional feed stream. In one embodiment, the light olefin stream is further separated and passed to a polymerization zone to produce a polyolefin stream.

Further provided in the invention is a process for the production of light olefins from a hydrocarbon feed stream. The process comprises providing the hydrocarbon feed stream, passing the hydrocarbon feed stream to a synthesis gas production zone to produce a synthesis gas stream, passing the synthesis gas stream to a carbon oxide conversion zone to produce a methanol stream, and passing the methanol stream to an oxygenate conversion zone to produce an olefin stream. The olefin stream is separated into component streams, one component stream comprised of a light olefin stream and another component stream comprised of a by-product stream. The by-product stream separated from the olefin stream is passed to a synthesis gas production zone to produce a synthesis gas stream, and the synthesis gas stream separated from the olefin stream is passed to a carbon oxide conversion zone to produce a methanol stream.

The synthesis gas production zones used in the process for the production of light olefins from a hydrocarbon feed stream can be the same or different. In other words, the process can use only one synthesis gas production zone for multiple steps of forming synthesis gas or more than one synthesis gas production zone can be used, with each hydrocarbon feed stream being sent to a different synthesis gas production zone. The same situation holds for one or more carbon oxide production zones, as well as one or more oxygenate conversion zones.

The hydrocarbon feed provided in the production of olefins is preferably a gas stream. In one embodiment, the hydrocarbon feed stream includes methane.

### DETAILED DESCRIPTION OF THE INVENTION

I. Integrated Gas to Olefins Process

This invention is directed to an integrated process for producing a light olefin product, particularly ethylene, propylene and optionally butylenes, from a hydrocarbon feed, which effectively recovers by-products for conversion into additional light olefin product. Preferably, the by-products produced in the process are separated from an olefin containing stream and converted to a synthesis gas (syngas) in a carbon oxide conversion zone for further conversion to desired product(s). By separating and converting undesirable by-products to more desirable olefin products, problems related to disposal and/or storage of potentially hazardous by-products is minimized.

In one embodiment of the invention, the hydrocarbon feed is passed to a synthesis gas production zone to produce a synthesis gas stream. The synthesis gas stream is preferably passed to a carbon oxide conversion zone to produce an oxygenate stream, preferably a methanol stream. The methanol stream is then passed to an oxygenate conversion zone to produce an olefin stream, and the olefin stream is separated into at least two component streams. At least one of the separated component streams is a by-product stream. Any or all of the by-product streams formed are passed to a synthesis gas production zone to produce synthesis gas, and the synthesis gas produced from the by-product(s) is ultimately converted to additional light olefin.

II. Production of Synthesis Gas from Hydrocarbon Feed Stream

A. Generally

The hydrocarbon feed stream from which the synthesis gas stream is produced according to this invention can be provided from any conventional source. For example, the hydrocarbon feed stream may include a natural or synthetic gas stream. Examples of sources of the hydrocarbon feed include biomass, natural gas, C₁-C₅ hydrocarbons, naphtha, heavy petroleum oils or coke (i.e., coal). Preferably, the hydrocarbon feed is a gas stream comprising methane in an amount of at least about 50% by volume, more preferably at least about 70% by volume, most preferably at least about 80% by volume, based on total volume of the hydrocarbon stream. In one embodiment of this invention, the hydrocarbon feed is a natural gas comprising at least 50% methane by volume.

In one embodiment of the invention, the hydrocarbon feed stream is first converted to synthesis gas. Any conventional synthesis gas production process can be used. Preferably, the hydrocarbon feed stream is converted to synthesis gas using a catalyst. In one embodiment of the invention, the catalyst is a nickel containing catalyst.

Synthesis gas comprises carbon monoxide and hydrogen. Optionally, carbon dioxide and nitrogen are included. Conventional processes for converting carbon components to syngas include steam reforming, partial oxidation, and autothermal reforming.

The hydrocarbon feed stream that is used in the conversion of hydrocarbon to synthesis gas, is optionally treated to remove impurities that can cause problems in further processing of the hydrocarbon feed stream. These impurities can poison many conventional propylene and ethylene forming catalysts. A majority of the impurities, which may be present, can be removed in any conventional manner. The hydrocarbon feed is preferably purified to remove sulfur compounds, nitrogen compounds, particulate matter, other condensables, and/or other potential catalyst poisons prior to being converted into synthesis gas.

In one embodiment of the invention, the hydrocarbon feed stream is passed to a synthesis gas production zone or synthesis gas plant. Synthesis gas refers to a combination of hydrogen and carbon oxide produced in a synthesis gas production zone from a hydrocarbon feed, the synthesis gas having an appropriate molar ratio of hydrogen to carbon oxide (carbon monoxide and/or carbon dioxide), as described below. The synthesis gas production zone can employ any conventional means of producing synthesis gas, including partial oxidation, steam or CO₂ reforming, or some combination of these two chemistries.

Steam reforming generally comprises contacting a hydrocarbon with steam to form synthesis gas. The process preferably includes the use of a catalyst.

Partial oxidation generally comprises contacting a hydrocarbon with oxygen or an oxygen containing gas such as air to form synthesis gas. Partial oxidation takes place with or without the use of a catalyst, although the use of a catalyst is preferred. In one embodiment, water (steam) is added with the feed in the partial oxidation process. Such an embodiment is generally referred to as autothermal reforming.

Conventional synthesis gas-generating processes include gas phase partial oxidation, autothermal reforming, fluid bed synthesis gas generation, catalytic partial oxidation and various processes for steam reforming.

B. Steam Reforming

In the catalytic steam reforming process, hydrocarbon feeds are converted to a mixture of H₂, CO and CO₂ by reacting hydrocarbons with steam over a catalyst. This process involves the following reactions:

CH₄+H₂O [symbol] CO+3H₂ (1)

or

CₙHₘ+nH₂O [symbol] nCO+[n+(m/2)]H₂ (2)

and

CO+H₂O [symbol] CO₂+H₂ (3) (shift reaction)

The reaction is carried out in the presence of a catalyst. Any conventional reforming type catalyst can be used. The catalyst used in the step of catalytic steam reforming comprises at least one active metal or metal oxide of Group 6 or Group 8 to 10 of the Periodic Table of the Elements. The Periodic Table of the Elements referred to herein is that from CRC Handbook of Chemistry and Physics, 82^{nd} Edition, 2001-2002, CRC Press LLC, which is incorporated herein by reference.

In one embodiment, the catalyst contains at least one Group 6 or Group 8-10 metal, or oxide thereof, having an atomic number of 28 or greater. Specific examples of reforming catalysts that can be used are nickel, nickel oxide, cobalt oxide, chromia and molybdenum oxide. Optionally, the catalyst is employed with least one promoter. Examples of promoters include alkali and rare earth promoters. Generally, promoted nickel oxide catalysts are preferred.

The amount of Group 6 or Group 8 to 10 metals in the catalyst can vary. Preferably, the catalyst includes from about 3 wt % to about 40 wt % of at least one Group 6 or Group 8 to 10 metal, based on total weight of the catalyst. Preferably, the catalyst includes from about 5 wt % to about 25 wt % of at least one Group 6 or Group 8 to 10 metal, based on total weight of the catalyst.

The reforming catalyst optionally contains one or more metals to suppress carbon deposition during steam reforming. Such metals are selected from the metals of Group 14 and Group 15 of the Periodic Table of the Elements. Preferred Group 14 and Group 15 metals include germanium, tin, lead, arsenic, antimony, and bismuth. Such metals are preferably included in the catalyst in an amount of from about 0.1 wt % to about 30 wt %, based on total weight of nickel in the catalyst.

In a catalyst comprising nickel and/or cobalt there may also be present one or more platinum group metals, which are capable of increasing the activity of the nickel and/or cobalt and of decreasing the tendency to carbon lay-down when reacting steam with hydrocarbons greater than methane. The concentration of such platinum group metal is typically in the range 0.0005 to 0.1% as metal, calculated as the whole catalyst unit. Further, the catalyst, especially in preferred forms, can contain a platinum group metal but no non-noble catalytic component. Such a catalyst is more suitable for the hydrocarbon steam reforming reaction than one containing a platinum group metal on a conventional support because a greater fraction of the active metal is accessible to the reacting gas. A typical content of platinum group metal when used alone is in the range 0.0005 to 0.5% w/w as metal, calculated on the whole catalytic unit.

In one embodiment, the reformer unit includes tubes which are packed with solid catalyst granules. Preferably, the solid catalyst granules comprise nickel or other catalytic agents deposited on a suitable inert carrier material. More preferably, the catalyst is NiO supported on calcium aluminate, alumina, spinel type magnesium aluminum oxide or calcium aluminate titanate.

In yet another embodiment, both the hydrocarbon feed stream and the steam are preheated prior to entering the reformer. The hydrocarbon feedstock is preheated up to as high a temperature as is consistent with the avoiding of undesired pyrolysis or other heat deterioration. Since steam reforming is endothermic in nature, and since there are practical limits to the amount of heat that can be added by indirect heating in the reforming zones, preheating of the feed is desired to facilitate the attainment and maintenance of a suitable temperature within the reformer itself. Accordingly, it is desirable to preheat both the hydrocarbon feed and the steam to a temperature of at least 200° C.; preferably at least 400° C. The reforming reaction is generally carried out at a reformer temperature of from about 500° C. to about 1,200° C., preferably from about 800° C. to about 1,100° C., and more preferably from about 900° C. to about 1,050° C.

Gas hourly space velocity in the reformer should be sufficient for providing the desired CO to CO₂ balance in the synthesis gas. Preferably, the gas hourly space velocity (based on wet feed) is from about 3,000 per hour to about 10,000 per hour, more preferably from about 4,000 per hour to about 9,000 per hour, and most preferably from about 5,000 per hour to about 8,000 per hour.

Any conventional reformer can be used in the step of catalytic steam reforming. The use of a tubular reformer is preferred. Preferably, the hydrocarbon feed is passed to a tubular reformer together with steam, and the hydrocarbon and steam contact a steam reforming catalyst. In one embodiment, the steam reforming catalyst is disposed in a plurality of furnace tubes that are maintained at an elevated temperature by radiant heat transfer and/or by contact with combustion gases. Fuel, such as a portion of the hydrocarbon feed, is burned in the reformer furnace to externally heat the reformer tubes therein. See, for example, Kirk-Othmer, Encyclopedia of Chemical Technology, 3rd Ed., 1990, vol. 12, p. 951; and Ullmann's Encyclopedia of Industrial Chemistry, 5th Ed., 1989, vol. A-12, p. 186, the relevant portions of each being fully incorporated herein by reference.

The ratio of steam to hydrocarbon feed will vary depending on the overall conditions in the reformer. The amount of steam employed is influenced by the requirement of avoiding carbon deposition on the catalyst, and by the acceptable methane content of the effluent at the reforming conditions maintained. On this basis, the mole ratio of steam to hydrocarbon feed in the conventional primary reformer unit is preferably from about 1.5:1 to about 5:1, preferably from about 2:1 to about 4:1.

The hydrogen to carbon oxide ratio of the synthesis gas produced will vary depending on the overall conditions of the reformer. Preferably, the molar ratio of hydrogen to carbon oxide in the synthesis gas will range from about 1:1 to about 5:1. More preferably the molar ratio of hydrogen to carbon oxide will range from about 2:1 to about 3:1. Even more preferably the molar ratio of hydrogen to carbon oxide will range from about 2:1 to about 2.5:1. Most preferably the molar ration of hydrogen to carbon oxide will range from about 2:1 to about 2.3:1.

Steam reforming is generally carried out at superatmospheric pressure. The specific operating pressure employed is influenced by the pressure requirements of the subsequent process in which the reformed gas mixture is to be employed. Although any superatmospheric pressure can be used in practicing the invention, pressures of from about 175 psig (1,308 kPa abs to about 1,100 psig (7,686 kPa abs are desirable. Preferably, steam reforming is carried out at a pressure of from about 300 psig (2,170 kPa abs.) to about 800 psig (5,687 kPa abs.), more preferably from about 350 psig (2,515 kPa abs.) to about 700 psig (4,928 kPa abs.).

C. Partial Oxidation

The invention further provides for the production of synthesis gas, or CO and H₂, by oxidative conversion (also referred to herein as partial oxidation) of hydrocarbon, particularly natural gas and C₁ to C₅ hydrocarbons. According to the process, hydrocarbon is reacted with free-oxygen to form the CO and H₂. The process is carried out with or without a catalyst. The use of a catalyst is preferred, preferably with the catalyst containing at least one non-transition or transition metal oxides. The process is essentially exothermic, and is an incomplete combustion reaction, having the following general formula:

CₙHₘ+(n/2)O₂ [symbol] nCO+(m/2)H₂ (4)

Non-catalytic partial oxidation of hydrocarbons to H₂, CO and CO₂ is desirably used for producing syngas from heavy fuel oils, primarily in locations where natural gas or lighter hydrocarbons, including naphtha, are unavailable or uneconomical compared to the use of fuel oil or crude oil. The non-catalytic partial oxidation process is carried out by injecting preheated hydrocarbon, oxygen and steam through a burner into a closed combustion chamber. Preferably, the individual components are introduced at a burner where they meet in a diffusion flame, producing oxidation products and heat. In the combustion chamber, partial oxidation of the hydrocarbons generally occurs with less than stoichiometric oxygen at very high temperatures and pressures. Preferably, the components are preheated and pressurized to reduce reaction time. The process preferably occurs at a temperature of from about 1,350° C. to about 1,600° C., and at a pressure of from above atmospheric to about 150 atm.

Catalytic partial oxidation comprises passing a gaseous hydrocarbon mixture, and oxygen, preferably in the form of air, over reduced or unreduced composite catalysts. The reaction is optionally accompanied by the addition of water vapor (steam). When steam is added, the reaction is generally referred to as autothermal reduction. Autothermal reduction is both exothermic and endothermic as a result of adding both oxygen and water.

In the catalytic partial oxidation process, the catalyst comprises at least one transition element selected from the group consisting of Ni, Co, Pd, Ru, Rh, Ir, Pt, Os and Fe. Preferably, the catalyst comprises at least one transition element selected from the group consisting of Pd, Pt, and Rh. In another embodiment, preferably the catalyst comprises at least one transition element selected form the group consisting of Ru, Rh, and Ir.

In one embodiment, the partial oxidation catalyst further comprises at least one metal selected from the group consisting of Ti, Zr, Hf, Y, Th, U, Zn, Cd, B, Al, Ti, Si, Sn, Pb, P, Sb, Bi, Mg, Ca, Sr, Ba, Ga, V, and Sc. Also, optionally included in the partial oxidation catalyst is at least one rare earth element selected from the group consisting of La, Ce, Pr, Nd, Pm, Sm, Eu, Gd, Tb, Dy, Ho, Er, Tm, Yb and Lu.

In another embodiment the catalyst employed in the process may comprise a wide range of catalytically active components, for example Pd, Pt, Rh, Ir, Os, Ru, Ni, Cr, Co, Ce, La and mixtures thereof. Materials not normally considered to be catalytically active may also be employed as catalysts, for example refractory oxides such as cordierite, mullite, mullite aluminium titanate, zirconia spinels and alumina.

In yet another embodiment, the catalyst is comprised of metals selected from those having atomic number 21 to 29, 40 to 47 and 72 to 79, the metals Sc, Ti V, Cr, Mn, Fe, Co, Ni, Cu, Zr, Nb, Mo, Tc, Ru, Rh, Pd, Ag, Hf, Ta, W, Re, Os Ir, Pt, and Au. The preferred metals are those in Group 8 of the Periodic Table of the Elements, that is Fe, Os, Co, Re, Ir, Pd, Pt, Ni, and Ru.

In another embodiment, the partial oxidation catalyst comprises at least one transition or non-transition metal deposited on a monolith support. The monolith supports are preferably impregnated with a noble metal such as Pt, Pd or Rh, or other transition metals such as Ni, Co, Cr and the like. Desirably, these monolith supports are prepared from solid refractory or ceramic materials such as alumina, zirconia, magnesia, ceria, silica, titania, mixtures thereof, and the like. Mixed refractory oxides, that is refractory oxides comprising at least two cations, may also be employed as carrier materials for the catalyst.

In one embodiment, the catalyst is retained in form of a fixed arrangement. The fixed arrangement generally comprises a fixed bed of catalyst particles. Alternatively, the fixed arrangement comprises the catalyst in the form of a monolith structure. The fixed arrangement may consist of a single monolith structure or, alternatively, may comprise a number of separate monolith structures combined to form the fixed arrangement. A preferred monolith structure comprises a ceramic foam. Suitable ceramic foams for use in the process are available commercially.

In yet another embodiment, the feed comprises methane, and the feed is injected with oxygen into the partial oxidation reformer at a methane to oxygen (i.e., O₂) ratio of from about 1.2:1 to about 10:1. Preferably the feed and oxygen are injected into the reformer at a methane to oxygen ratio of from about 1.6:1 to about 8:1, more preferably from about 1.8:1 to about 4:1.

Water may or may not be added to the partial oxidation process. When added, the concentration of water injected into the reformer is not generally greater than about 65 mole %, based on total hydrocarbon and water feed content. Preferably, when water is added, it is added at a water to methane ratio of not greater than 3:1, preferably not greater than 2:1.

The catalyst may or may not be reduced before the catalytic reaction. In one embodiment, the catalyst is reduced and reduction is carried out by passing a gaseous mixture comprising hydrogen and inert gas (e.g., N₂, He, or Ar) over the catalyst in a fixed bed reactor at a catalyst reduction pressure of from about 1 atm to about 5 atm, and a catalyst reduction temperature of from about 300° C. to about 700° C. Hydrogen gas is used as a reduction gas, preferably at a concentration of from about 1 mole % to about 100 mole %, based on total amount of reduction gas. Desirably, the reduction is further carried out at a space velocity of reducing gas mixture of from about 103 cm³/g hr to about 105 cm³/g hr for a period of from about 0.5 hour to about 20 hours.

In one embodiment, the partial oxidation catalyst is not reduced by hydrogen. When the catalyst is not reduced by hydrogen before the catalytic reaction, the reduction of the catalyst can be effected by passing the hydrocarbon feed and oxygen (or air) over the catalyst at temperature in the range of from about 500° C. to about 900° C. for a period of from about 0.1 hour to about 10 hours.

In the partial oxidation process, carbon monoxide (CO) and hydrogen (H₂) are formed as major products, and water and carbon dioxide (CO₂) as minor products. The gaseous product stream comprises the above mentioned products, unconverted reactants (i.e. methane or natural gas and oxygen) and components of feed other than reactants.

When water is added in the feed, the H₂:CO mole ratio in the product is increased by the shift reaction: CO+H₂O [symbol] H₂+CO₂. This reaction occurs simultaneously with the oxidative conversion of the hydrocarbon in the feed to CO and H₂ or synthesis gas. The hydrocarbon used as feed in the partial oxidation process is preferably in the gaseous phase when contacting the catalyst. The partial oxidation process is particularly suitable for the partial oxidation of methane, natural gas, associated gas or other sources of light hydrocarbons. In this respect, the term “light hydrocarbons” is a reference to hydrocarbons having from 1 to 5 carbon atoms. The process may be advantageously applied in the conversion of gas from naturally occurring reserves of methane, which contain substantial amounts of carbon dioxide. In one embodiment, the hydrocarbon feed preferably contains from about 10 mole % to about 90 mole % methane, based on total feed content. More preferably, the hydrocarbon feed contains from about 20 mole % to about 80 mole % methane, based on total feed content. In another embodiment, the feed comprises methane in an amount of at least 50% by volume, more preferably at least 70% by volume, and most preferably at least 80% by volume.

In one embodiment of the invention, the hydrocarbon feedstock is contacted with the catalyst in a mixture with an oxygen-containing gas. Air is suitable for use as the oxygen-containing gas. Substantially pure oxygen as the oxygen-containing gas is preferred on occasions where there is a need to avoid handling large amounts of inert gas such as nitrogen. The feed optionally comprises steam.

In another embodiment of the invention, the hydrocarbon feedstock and the oxygen-containing gas are preferably present in the feed in such amounts as to give an oxygen-to-carbon ratio in the range of from about 0.3:1 to about 0.8:1, more preferably, in the range of from about 0.45:1 to about 0.75:1. References herein to the oxygen-to-carbon ratio refer to the ratio of oxygen in the from of oxygen molecules (O₂) to carbon atoms present in the hydrocarbon feedstock. Preferably, the oxygen-to-carbon ratio is in the range of from about 0.45:1 to about 0.65:1, with oxygen-to-carbon ratios in the region of the stoichiometric ratio of 0.5:1, that is ratios in the range of from about 0.45:1 to about 0.65:1, being more preferred. When steam is present in the feed, the steam-to-carbon ratio is not greater than about 3.0:1, more preferably not greater than about 2.0:1. The hydrocarbon feedstock, the oxygen-containing gas and steam, if present, are preferably well mixed prior to being contacted with the catalyst.

The partial oxidation process is operable over a wide range of pressures. For applications on a commercial scale, elevated pressures, that is pressures significantly above atmospheric pressure, are preferred. In one embodiment, the partial oxidation process is operated at pressures of greater than atmospheric up to about 150 bars. Preferably, the partial oxidation process is operated at a pressure in the range of from about 2 bars to about 125 bars, more preferably from about 5 bars to about 100 bars.

The partial oxidation process is also operable over a wide range of temperatures. At commercial scale, the feed is preferably contacted with the catalyst at high temperatures. In one embodiment, the feed mixture is contacted with the catalyst at a temperature in excess of 600° C. Preferably, the feed mixture is contacted with the catalyst at a temperature in the range of from about 600° C. to about 1,700° C., more preferably from about 800° C. to about 1,600° C. The feed mixture is preferably preheated prior to contacting the catalyst.

The feed is provided during the operation of the process at a suitable space velocity to form a substantial amount of CO in the product. In one embodiment, gas space velocities (expressed in normal liters of gas per kilogram of catalyst per hour) are in the range of from about 20,000 Nl/kg/hr to about 100,000,000 Nl/kg/hr, more preferably in the range of from about 50,000 Nl/kg/hr to about 50,000,000 Nl/kg/hr, and most preferably in the range of from about 500,000 Nl/kg/hr to about 30,000,000 Nl/kg/hr.

D. Combination Processes

Combination reforming processes can also be incorporated into this invention. Examples of combination reforming processes include autothermal reforming and fixed bed syngas generation. These processes involve a combination of gas phase partial oxidation and steam reforming chemistry.

The autothermal reforming process preferably comprises two synthesis gas generating processes, a primary oxidation process and a secondary steam reforming process. In one embodiment, a hydrocarbon feed stream is steam reformed in a tubular primary reformer by contacting the hydrocarbon and steam with a reforming catalyst to form a hydrogen and carbon monoxide containing primary reformed gas, the carbon monoxide content of which is further increased in the secondary reformer. In one embodiment, the secondary reformer includes a cylindrical refractory lined vessel with a gas mixer, preferably in the form of a burner in the inlet portion of the vessel and a bed of nickel catalyst in the lower portion. In a more preferred embodiment, the exit gas from the primary reformer is mixed with air and residual hydrocarbons, and the mixed gas partial oxidized to carbon monoxides.

In another embodiment incorporating the autothermal reforming process, partial oxidation is carried out as the primary oxidating process. Preferably, hydrocarbon feed, oxygen, and optionally steam, are heated and mixed at an outlet of a single large coaxial burner or injector which discharges into a gas phase partial oxidation zone. Oxygen is preferably supplied in an amount which is less than the amount required for complete combustion.

Upon reaction in the partial oxidation combustion zone, the gases flow from the primary reforming process into the secondary reforming process. In one embodiment, the gases are passed over a bed of steam reforming catalyst particles or a monolithic body, to complete steam reforming. Desirably, the entire hydrocarbon conversion is completed by a single reactor aided by internal combustion.

In an alternative embodiment of the invention, a fixed bed syngas generation process is used to form synthesis gas. In the fixed bed syngas generation process, hydrocarbon feed and oxygen or an oxygen-containing gas are introduced separately into a fluid catalyst bed. Preferably, the catalyst is comprised of nickel and supported primarily on alpha alumina.

The fixed bed syngas generation process is carried out at conditions of elevated temperatures and pressures that favor the formation of hydrogen and carbon monoxide when, for example, methane is reacted with oxygen and steam. Preferably, temperatures are in excess of about 1,700° F. (927° C.), but not so high as to cause disintegration of the catalyst or the sticking of catalyst particles together. Preferably, temperatures range from about 1,750° F. (954° C.) to about 1,950° F. (1,066° C.), more preferably, from about 1,800° F. (982° C.) to about 1,850° F. (1,010° C.).

Pressure in the fixed bed syngas generation process may range from atmospheric to about 40 atmospheres. In one embodiment, pressures of from about 20 atmospheres to about 30 atmospheres are preferred, which allows subsequent processes to proceed without intermediate compression of product gases.

In one embodiment of the invention, methane, steam, and oxygen are introduced into a fluid bed by separately injecting the methane and oxygen into the bed. Alternatively, each stream is diluted with steam as it enters the bed. Preferably, methane and steam are mixed at a methane to steam molar ratio of from about 1:1 to about 3:1, and more preferably from about 1.5:1 to about 2.5:1, and the methane and steam mixture is injected into the bed. Preferably, the molar ratio of oxygen to methane is from about 0.2:1 to about 1.0:1, more preferably from about 0.4:1 to about 0.6:1.

In another embodiment of the invention, the fluid bed process is used with a nickel based catalyst supported on alpha alumina. In another embodiment, silica is included in the support. The support is preferably comprised of at least 95 wt % alpha alumina, more preferably at least about 98% alpha alumina, based on total weight of the support.

In one embodiment, a gaseous mixture of hydrocarbon feedstock and oxygen-containing gas are contacted with a reforming catalyst under adiabatic conditions. For the purposes of this invention, the term “adiabatic” refers to reaction conditions in which substantially all heat loss and radiation from the reaction zone are prevented, with the exception of heat leaving in the gaseous effluent stream of the reactor.

III. Production of Oxygenate from Synthesis Gas

The synthesis gas is sent to an oxygenate synthesis process (i.e., a carbon oxide conversion process) and converted to an oxygenate composition. Conventional processes can be used. Preferably, the synthesis gas is sent to a methanol synthesis gas process for converting into a methanol composition, which optionally includes other oxygenates. The methanol synthesis gas process is accomplished in the presence of a methanol synthesis catalyst.

In one embodiment, the synthesis gas is sent “as is” to the methanol synthesis process. In another embodiment, the hydrogen, carbon monoxide, and/or carbon dioxide content of the synthesis gas is adjusted for efficiency of conversion. Desirably, the synthesis gas input to the methanol synthesis reactor has a molar ratio of hydrogen (H₂) to carbon oxides (CO+CO₂) in the range of from about 0.5:1 to about 20:1, preferably in the range of from about 2:1 to about 10:1. In another embodiment, the synthesis gas has a molar ratio of hydrogen (H₂) to carbon monoxide (CO) of at least 2:1. Carbon dioxide is optionally present in an amount of not greater than 50% by weight, based on total weight of the synthesis gas.

Desirably, the stoichiometric molar ratio is sufficiently high so as maintain a high yield of methanol, but not so high as to reduce the volume productivity of methanol. Preferably, the synthesis gas fed to the methanol synthesis process has a stoichiometric molar ratio (i.e., a molar ratio of H₂:(2CO+3CO₂)) of from about 1.0:1 to about 2.7:1, more preferably from about 1.1 to about 2.0, more preferably a stoichiometric molar ratio of from about 1.2:1 to about 1.8:1.

The CO₂ content, relative to that of CO, in the synthesis gas should be high enough so as to maintain an appropriately high reaction temperature and to minimize the amount of undesirable by-products such as paraffins. At the same time, the relative CO₂ to CO content should not be too high so as to reduce methanol yield. Desirably, the synthesis gas contains CO₂ and CO at a ratio of from about 0.5 to about 1.2, preferably from about 0.6 to about 1.0.

In one embodiment, the catalyst used in the methanol synthesis process includes an oxide of at least one element selected from the group consisting of copper, silver, zinc, boron, magnesium, aluminum, vanadium, chromium, manganese, gallium, palladium, osmium and zirconium. Preferably, the catalyst is a copper based catalyst, more preferably in the form of copper oxide.

In another embodiment, the catalyst used in the methanol synthesis process is a copper based catalyst, which includes an oxide of at least one element selected from the group consisting of silver, zinc, boron, magnesium, aluminum, vanadium, chromium, manganese, gallium, palladium, osmium and zirconium. Preferably, the catalyst contains copper oxide and an oxide of at least one element selected from the group consisting of zinc, magnesium, aluminum, chromium, and zirconium. More preferably, the catalyst contains oxides of copper and zinc.

In yet another embodiment, the methanol synthesis catalyst comprises copper oxide, zinc oxide, and at least one other oxide. Preferably, the at least one other oxide is selected from the group consisting of zirconium oxide, chromium oxide, vanadium oxide, magnesium oxide, aluminum oxide, titanium oxide, hafnium oxide, molybdenum oxide, tungsten oxide, and manganese oxide.

In various embodiments, the methanol synthesis catalyst comprises from about 10 wt % to about 70 wt % copper oxide, based on total weight of the catalyst. Preferably, the methanol synthesis contains from about 15 wt % to about 68 wt % copper oxide, and more preferably from about 20 wt % to about 65 wt % copper oxide, based on total weight of the catalyst.

In one embodiment, the methanol synthesis catalyst comprises from about 3 wt % to about 30 wt % zinc oxide, based on total weight of the catalyst. Preferably, the methanol synthesis catalyst comprises from about 4 wt % to about 27 wt % zinc oxide, more preferably from about 5 wt % to about 24 wt % zinc oxide.

In embodiments in which copper oxide and zinc oxide are both present in the methanol synthesis catalyst, the ratio of copper oxide to zinc oxide can vary over a wide range. Preferably in such embodiments, the methanol synthesis catalyst comprises copper oxide and zinc oxide in a Cu:Zn atomic ratio of from about 0.5:1 to about 20:1, preferably from about 0.7:1 to about 15:1, more preferably from about 0.8:1 to about 5:1.

The methanol synthesis catalyst is made according to conventional processes. Examples of such processes can be found in U.S. Pat. Nos. 6,114,279; 6,054,497; 5,767,039; 5,045,520; 5,254,520; 5,610,202; 4,666,945; 4,455,394; 4,565,803; 5,385,949, with the descriptions of each being fully incorporated herein by reference.

In one embodiment, the synthesis gas formed in the synthesis gas conversion plant is cooled prior to sending to the methanol synthesis reactor. Preferably, the synthesis gas is cooled so as to condense at least a portion of the water vapor formed during the synthesis gas process.

The methanol synthesis process used to manufacture the methanol composition of this invention can be any conventional process. Examples of such processes include batch processes and continuous processes. Continuous processes are preferred. Tubular bed processes and fluidized bed processes are particularly preferred types of continuous processes.

In general, the methanol synthesis process takes place according to the following reactions:

CO+2H₂→CH₃OH (4)

CO₂+3H₂→CH₃OH+H₂O (5)

The methanol synthesis process is effective over a wide range of temperatures. In one embodiment, the synthesis gas is contacted with the methanol synthesis catalyst at a temperature in the range of from about 150° C. to about 450° C., preferably in a range of from about 175° C. to about 350° C., more preferably in a range of from about 200° C. to about 300° C.

The process is also operable over a wide range of pressures. In one embodiment, the synthesis gas is contacted with the methanol synthesis catalyst at a pressure in the range of from about 15 atmospheres to about 125 atmospheres, preferably in a range of from about 20 atmospheres to about 100 atmospheres, more preferably in a range of from about 25 atmospheres to about 75 atmospheres.

Gas hourly space velocities vary depending upon the type of continuous process that is used. Desirably, gas hourly space velocity of flow of gas through the catalyst bed is in the range of from about 50 hr⁻¹ to about 50,000 hr⁻¹. Preferably, gas hourly space velocity of flow of gas through the catalyst bed is in the range of from about 250 hr⁻¹ to about 25,000 hr⁻¹, more preferably from about 500 hr⁻¹ to about 10,000 hr⁻¹.

In one embodiment of the invention, crude methanol is produced from the methanol synthesis process. The crude methanol is then processed to form a methanol feed. Preferably, the methanol feed is of sufficiently high quality to use a feed in a catalytic methanol conversion reaction to form light olefins, particularly substantial amounts of ethylene and propylene.

Processing of the crude methanol is accomplished by any conventional means. Examples of such means include distillation, selective condensation, and selective adsorption. Process conditions, e.g., temperatures and pressures, can vary according to the particular methanol composition desired. It is particularly desirable to minimize the amount of water and light boiling point components in the methanol composition, but without substantially reducing the amount of methanol and desirable aldehydes and/or other desirable alcohols also present.

In one embodiment, the crude methanol product from the methanol synthesis reactor is further treated to reduce water content and other undesirable impurities prior to converting to olefin product. Conventional treatment processes can be used. Examples of such processes include distillation, selective condensation, and selective adsorption.

In one embodiment, a crude methanol stream comprising methanol, dimethyl ether, fusel oils (i.e., hydrocarbons having a boiling point greater than methanol), and water is withdrawn from a carbon oxide conversion zone. The crude methanol stream is then passed to a distillation column, conventionally referred to as a topping column. Desirably, the topping column operates at a pressure of from about 20 kPa to about 200 kPa. Preferably, the topping column operates at a pressure of from about 25 kPa to about 150 kPa, more preferably from about 30 kPa to about 125 kPa, and most preferably from about 40 kPa to about 100 kPa.

A first light ends stream is removed from an upper portion of the topping column. Preferably, the lights ends stream contains dissolved gases (e.g., hydrogen, methane, carbon oxides, and nitrogen), and light ends (e.g., ethers, ketones, and aldehydes). In one embodiment of the invention, the dissolved gases, the light ends, or both are used as fuel. In another embodiment, the dissolved gases, light ends, or both are sent to a synthesis gas production zone to produce additional synthesis gas, which can ultimately be converted to additional methanol, preferably further converted to olefin(s).

A bottoms stream is preferably removed from a lower portion of the topping column, and passed to a second distillation column, conventionally referred to as a refining column. From the refining column, a second light ends stream is withdrawn, preferably at an upper portion of the refining column. In one embodiment, the second light ends stream is combined with the first light ends stream from the topping column to form a combined purge stream. The combined purge stream is preferably used for fuel.

The refining column operates at a pressure of from about 0.5 atm to about 10 atm. Preferably, the refining column operates at a pressure of from about 0.6 to about 5 atm, more preferably from about 0.7 to about 3 atm, and most preferably from about 0.7 to about 2 atm. The refining column is used to further separate methanol from water and fusel oils, which remain in the bottoms stream of the topping column, so as to provide a high purity methanol stream, a fusel oil stream, and a water stream.

The methanol stream separated from the refining column is suitable for use in any system that uses methanol as a feedstream. Preferably, the methanol is suitable for use in an oxygenate conversion system.

In one embodiment, the methanol stream separated from the refining column comprises at least 98 wt % methanol, based on total weight of the methanol stream. Preferably, the methanol stream comprises at least 98.5 wt % methanol, more preferably at least 99.0 wt % methanol, and most preferably at least 99.5 wt % methanol, based on total weight of the methanol stream.

In another embodiment, the methanol stream separated from the refining column comprises less than 0.2 wt % water, based on total weight of the methanol stream. Preferably, the methanol stream comprises less than 0.15 wt % water, more preferably less than 0.1 wt % water, and most preferably less than 0.05 wt % water, based on total weight of the methanol stream.

In yet another embodiment, the methanol stream separated from the refining column comprises less than 40 wppm acetone, based on total weight of the methanol stream. Preferably the methanol stream separated from the refining column comprises less than 30 wppm acetone, more preferably less than 25 wt % acetone, and most preferably less than 20 wt % acetone, based on total weight of the methanol stream.

IV. Production of Olefin Products from Oxygenate

A. General Process Description

In one embodiment of the invention, an oxygenate composition is converted to olefins by contacting the oxygenate composition with an olefin forming catalyst to form the olefin product. Preferably, the oxygenate composition is a crude methanol stream from the carbon oxide conversion zone or a refined or treated methanol stream recovered from the crude methanol stream. The olefin product is recovered, and water, which forms during the conversion of the oxygenates in the methanol to olefins, is removed. After removing the water, the olefins are separated into individual olefin streams, and each individual olefin stream is available for further processing.

B. Adding Other Oxygenates to Methanol Composition

In an optional embodiment of this invention, a methanol composition from a methanol synthesis process is converted to olefin along with other oxygenates or diluents. The additional oxygenates or diluents can be co-mixed with the methanol composition or added as a separate feed stream to an oxygenate conversion reactor. In one embodiment, the additional oxygenate is one or more alcohol(s), preferably aliphatic alcohol(s) where the aliphatic moiety of the alcohol(s) has from 1 to 10 carbon atoms, preferably from 1 to 5 carbon atoms, and most preferably from 1 to 4 carbon atoms. Ethanol is most preferred. The alcohols include lower straight and branched chain aliphatic alcohols and their unsaturated counterparts. Non-limiting examples of oxygenates include ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof. In the most preferred embodiment, the feedstock is selected from one or more of methanol, ethanol, dimethyl ether, diethyl ether or a combination thereof, more preferably methanol and dimethyl ether, and most preferably methanol.

The methanol feed stream, in one embodiment, contains one or more diluent(s), typically used to reduce the concentration of the methanol, and are generally non-reactive to the oxygenates in the composition or to the molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred.

The diluent is either added directly to the methanol feedstock entering into a reactor or added directly into a reactor, or added with a molecular sieve catalyst composition. In one embodiment, the amount of diluent in the feedstock is in the range of from about 1 to about 99 mole percent based on the total number of moles of the feedstock and diluent, preferably from about 1 to 80 mole percent, more preferably from about 5 to about 50 more percent, most preferably from about 5 to about 25 mole percent. In one embodiment, other hydrocarbons are added to the feedstock either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

C. Description of Olefin Forming Catalyst

Any catalyst capable of converting oxygenate to olefin can be used in this invention. Molecular sieve catalysts are preferred. Examples of such catalysts include zeolite as well as non-zeolite molecular sieves, and are of the large, medium or small pore type. Non-limiting examples of these molecular sieves are the small pore molecular sieves, AEI, AFT, APC, ATN, ATT, ATV, AWW, BIK, CAS, CHA, CHI, DAC, DDR, EDI, ER1, GOO, KFI, LEV, LOV, LTA, MON, PAU, PHI, RHO, ROG, THO, and substituted forms thereof; the medium pore molecular sieves, AFO, AEL, EUO, HEU, FER, MEL, MFI, MTW, MTT, TON, and substituted forms thereof; and the large pore molecular sieves, EMT, FAU, and substituted forms thereof. Other molecular sieves include ANA, BEA, CFI, CLO, DON, GIS, LTL, MER, MOR, MWW and SOD. Non-limiting examples of the preferred molecular sieves, particularly for converting an oxygenate containing feedstock into olefin(s), include AEL, AFY, BEA, CHA, EDI, FAU, FER, GIS, LTA, LTL, MER, MFI, MOR, MTT, MWW, TAM and TON. In one preferred embodiment, the molecular sieve of the invention has an AEI topology or a CHA topology, or a combination thereof, most preferably a CHA topology.

Molecular sieve materials all have 3-dimensional, four-connected framework structure of corner-sharing TO₄ tetrahedra, where T is any tetrahedrally coordinated cation. These molecular sieves are typically described in terms of the size of the ring that defines a pore, where the size is based on the number of T atoms in the ring. Other framework-type characteristics include the arrangement of rings that form a cage, and when present, the dimension of channels, and the spaces between the cages. See van Bekkum, et al., Introduction to Zeolite Science and Practice, Second Completely Revised and Expanded Edition, Volume 137, pages 1-67, Elsevier Science, B.V., Amsterdam, Netherlands (2001).

The small, medium and large pore molecular sieves have from a 4-ring to a 12-ring or greater framework-type. In a preferred embodiment, the molecular sieves have 8-, 10- or 12-ring structures or larger and an average pore size in the range of from about 3 Å to 15 Å. In the most preferred embodiment, the molecular sieves of the invention, preferably silicoaluminophosphate molecular sieves, have 8-rings and an average pore size less than about 5 Å, preferably in the range of from 3 Å to about 5 Å, more preferably from 3 Å to about 4.5 Å, and most preferably from 3.5 Å to about 4.2 Å.

Molecular sieves, particularly zeolitic and zeolitic-type molecular sieves, preferably have a molecular framework of one, preferably two or more corner-sharing [TO₄] tetrahedral units, more preferably, two or more [SiO₄], [AlO₄] and/or [PO₄] tetrahedral units, and most preferably [SiO₄], [AlO₄] and [PO₄] tetrahedral units. These silicon, aluminum, and phosphorous based molecular sieves and metal containing silicon, aluminum and phosphorous based molecular sieves have been described in detail in numerous publications including for example, U.S. Pat. No. 4,567,029 (MeAPO where Me is Mg, Mn, Zn, or Co), U.S. Pat. No. 4,440,871 (SAPO), European Patent Application EP-A-0 159 624 (ELAPSO where El is As, Be, B, Cr, Co, Ga, Ge, Fe, Li, Mg, Mn, Ti or Zn), U.S. Pat. No. 4,554,143 (FeAPO), U.S. Pat. Nos. 4,822,478, 4,683,217, 4,744,885 (FeAPSO), EP-A-0 158 975 and U.S. Pat. No. 4,935,216 (ZnAPSO, EP-A-0 161 489 (CoAPSO), EP-A-0 158 976 (ELAPO, where EL is Co, Fe, Mg, Mn, Ti or Zn), U.S. Pat. No. 4,310,440 (AlPO₄), EP-A-0 158 350 (SENAPSO), U.S. Pat. No. 4,973,460 (LiAPSO), U.S. Pat. No. 4,789,535 (LiAPO), U.S. Pat. No. 4,992,250 (GeAPSO), U.S. Pat. No. 4,888,167 (GeAPO), U.S. Pat. No. 5,057,295 (BAPSO), U.S. Pat. No. 4,738,837 (CrAPSO), U.S. Pat. Nos. 4,759,919, and 4,851,106 (CrAPO), U.S. Pat. Nos. 4,758,419, 4,882,038, 5,434,326 and 5,478,787 (MgAPSO), U.S. Pat. No. 4,554,143 (FeAPO), U.S. Pat. No. 4,894,213 (AsAPSO), U.S. Pat. No. 4,913,888 (AsAPO), U.S. Pat. Nos. 4,686,092, 4,846,956 and 4,793,833 (MnAPSO), U.S. Pat. Nos. 5,345,011 and 6,156,931 (MnAPO), U.S. Pat. No. 4,737,353 (BeAPSO), U.S. Pat. No. 4,940,570 (BeAPO), U.S. Pat. Nos. 4,801,309, 4,684,617 and 4,880,520 (TiAPSO), U.S. Pat. Nos. 4,500,651, 4,551,236 and 4,605,492 (TiAPO), U.S. Pat. Nos. 4,824,554, 4,744,970 (CoAPSO), U.S. Pat. No. 4,735,806 (GaAPSO) EP-A-0 293 937 (QAPSO, where Q is framework oxide unit [QO₂]), as well as U.S. Pat. Nos. 4,567,029, 4,686,093, 4,781,814, 4,793,984, 4,801,364, 4,853,197, 4,917,876, 4,952,384, 4,956,164, 4,956,165, 4,973,785, 5,241,093, 5,493,066 and 5,675,050, all of which are herein fully incorporated by reference.

Other molecular sieves include those described in EP-0 888 187 B 1 (microporous crystalline metallophosphates, SAPO₄ (UIO-6)), U.S. Pat. No. 6,004,898 (molecular sieve and an alkaline earth metal), U.S. patent application Ser. No. 09/511,943 filed Feb. 24, 2000 (integrated hydrocarbon co-catalyst), PCT WO 01/64340 published Sep. 7, 2001 (thorium containing molecular sieve), and R. Szostak, Handbook of Molecular Sieves, Van Nostrand Reinhold, New York, N.Y. (1992), which are all herein fully incorporated by reference.

The more preferred silicon, aluminum and/or phosphorous containing molecular sieves, and aluminum, phosphorous, and optionally silicon, containing molecular sieves include aluminophosphate (ALPO) molecular sieves and silicoaluminophosphate (SAPO) molecular sieves and substituted, preferably metal substituted, ALPO and SAPO molecular sieves. The most preferred molecular sieves are SAPO molecular sieves, and metal substituted SAPO molecular sieves. In an embodiment, the metal is an alkali metal of Group IA of the Periodic Table of Elements, an alkaline earth metal of Group IIA of the Periodic Table of Elements, a rare earth metal of Group IIIB, including the Lanthanides: lanthanum, cerium, praseodymium, neodymium, samarium, europium, gadolinium, terbium, dysprosium, holmium, erbium, thulium, ytterbium and lutetium; and scandium or yttrium of the Periodic Table of Elements, a transition metal of Groups IVB, VB, VIIB, VIIB, VIIIB, and IB of the Periodic Table of Elements, or mixtures of any of these metal species. In one preferred embodiment, the metal is selected from the group consisting of Co, Cr, Cu, Fe, Ga, Ge, Mg, Mn, Ni, Sn, Ti, Zn and Zr, and mixtures thereof. In another preferred embodiment, these metal atoms discussed above are inserted into the framework of a molecular sieve through a tetrahedral unit, such as [MeO₂], and carry a net charge depending on the valence state of the metal substituent. For example, in one embodiment, when the metal substituent has a valence state of +2, +3, +4, +5, or +6, the net charge of the tetrahedral unit is between −2 and +2.

In one embodiment, the molecular sieve, as described in many of the U.S. Patents mentioned above, is represented by the empirical formula, on an anhydrous basis:

mR:(MₓAl_{y}P_{z})O₂

wherein R represents at least one templating agent, preferably an organic templating agent; m is the number of moles of R per mole of (MₓAl_{y}P_{z})O₂ and m has a value from 0 to 1, preferably 0 to 0.5, and most preferably from 0 to 0.3; x, y, and z represent the mole fraction of Al, P and M as tetrahedral oxides, where M is a metal selected from one of Group IA, IIA, IB, IIIB, IVB, VB, VIIB, VIIB, VIIIB and Lanthanide's of the Periodic Table of Elements, preferably M is selected from one of the group consisting of Co, Cr, Cu, Fe, Ga, Ge, Mg, Mn, Ni, Sn, Ti, Zn and Zr. In an embodiment, m is greater than or equal to 0.2, and x, y and z are greater than or equal to 0.01.

In another embodiment, m is greater than 0.1 to about 1, x is greater than 0 to about 0.25, y is in the range of from 0.4 to 0.5, and z is in the range of from 0.25 to 0.5, more preferably m is from 0.15 to 0.7, x is from 0.01 to 0.2, y is from 0.4 to 0.5, and z is from 0.3 to 0.5.

Non-limiting examples of SAPO and ALPO molecular sieves used in the invention include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44 (U.S. Pat. No. 6,162,415), SAPO-47, SAPO-56, ALPO-5, ALPO-11, ALPO-18, ALPO-31, ALPO-34, ALPO-36, ALPO-37, ALPO-46, and metal containing molecular sieves thereof. The more preferred zeolite-type molecular sieves include one or a combination of SAPO-18, SAPO-34, SAPO-35, SAPO-44, SAPO-56, ALPO-18 and ALPO-34, even more preferably one or a combination of SAPO-18, SAPO-34, ALPO-34 and ALPO-18, and metal containing molecular sieves thereof, and most preferably one or a combination of SAPO-34 and ALPO-18, and metal containing molecular sieves thereof.

In an embodiment, the molecular sieve is an intergrowth material having two or more distinct phases of crystalline structures within one molecular sieve composition. In particular, intergrowth molecular sieves are described in the U.S. patent application Ser. No. 09/924,016 filed Aug. 7, 2001 and PCT WO 98/15496 published Apr. 16, 1998, both of which are herein fully incorporated by reference. In another embodiment, the molecular sieve comprises at least one intergrown phase of AEI and CHA framework-types. For example, SAPO-18, ALPO-18 and RUW-18 have an AEI framework-type, and SAPO-34 has a CHA framework-type.

In one embodiment, the molecular sieves used in the invention are combined with one or more other molecular sieves. In another embodiment, the preferred silicoaluminophosphate or aluminophosphate molecular sieves, or a combination thereof, are combined with one more of the following non-limiting examples of molecular sieves described in the following: Beta (U.S. Pat. No. 3,308,069), ZSM-5 (U.S. Pat. Nos. 3,702,886, 4,797,267 and 5,783,321), ZSM-11 (U.S. Pat. No. 3,709,979), ZSM-12 (U.S. Pat. No. 3,832,449), ZSM-12 and ZSM-38 (U.S. Pat. No. 3,948,758), ZSM-22 (U.S. Pat. No. 5,336,478), ZSM-23 (U.S. Pat. No. 4,076,842), ZSM-34 (U.S. Pat. No. 4,086,186), ZSM-35 (U.S. Pat. No. 4,016,245, ZSM-48 (U.S. Pat. No. 4,397,827), ZSM-58 (U.S. Pat. No. 4,698,217), MCM-1 (U.S. Pat. No. 4,639,358), MCM-2 (U.S. Pat. No. 4,673,559), MCM-3 (U.S. Pat. No. 4,632,811), MCM-4 (U.S. Pat. No. 4,664,897), MCM-5 (U.S. Pat. No. 4,639,357), MCM-9 (U.S. Pat. No. 4,880,611), MCM-10 (U.S. Pat. No. 4,623,527), MCM-14 (U.S. Pat. No. 4,619,818), MCM-22 (U.S. Pat. No. 4,954,325), MCM-41 (U.S. Pat. No. 5,098,684), M-41S (U.S. Pat. No. 5,102,643), MCM-48 (U.S. Pat. No. 5,198,203), MCM-49 (U.S. Pat. No. 5,236,575), MCM-56 (U.S. Pat. No. 5,362,697), ALPO-11 (U.S. Pat. No. 4,310,440), titanium aluminosilicates (TASO), TASO-45 (EP-A-0 229,295), boron silicates (U.S. Pat. No. 4,254,297), titanium aluminophosphates (TAPO) (U.S. Pat. No. 4,500,651), mixtures of ZSM-5 and ZSM-11 (U.S. Pat. No. 4,229,424), ECR-18 (U.S. Pat. No. 5,278,345), SAPO-34 bound ALPO-5 (U.S. Pat. No. 5,972,203), PCT WO 98/57743 published Dec. 23, 1988 (molecular sieve and Fischer-Tropsch), U.S. Pat. No. 6,300,535 (MFI-bound zeolites), and mesoporous molecular sieves (U.S. Pat. Nos. 6,284,696, 5,098,684, 5,102,643 and 5,108,725), which are all herein fully incorporated by reference.

The molecular sieves are made or formulated into catalysts by combining the synthesized molecular sieves with a binder and/or a matrix material to form a molecular sieve catalyst composition or a formulated molecular sieve catalyst composition. This formulated molecular sieve catalyst composition is formed into useful shape and sized particles by conventional techniques such as spray drying, pelletizing, extrusion, and the like.

There are many different binders that are useful in forming the molecular sieve catalyst composition. Non-limiting examples of binders that are useful alone or in combination include various types of hydrated alumina, silicas, and/or other inorganic oxide sol. One preferred alumina containing sol is aluminum chlorhydrol. The inorganic oxide sol acts like glue binding the synthesized molecular sieves and other materials such as the matrix together, particularly after thermal treatment. Upon heating, the inorganic oxide sol, preferably having a low viscosity, is converted into an inorganic oxide matrix component. For example, an alumina sol will convert to an aluminum oxide matrix following heat treatment.

Aluminum chlorhydrol, a hydroxylated aluminum based sol containing a chloride counter ion, has the general formula of AlₘOₙ(OH)ₒClₚ.x(H₂O) wherein m is 1 to 20, n is 1 to 8, o is 5 to 40, p is 2 to 15, and x is 0 to 30. In one embodiment, the binder is Al₁₃O₄(OH)₂₄C₇.I2(H₂O) as is described in G. M. Wolterman, et al., Stud. Surf. Sci. and Catal., 76, pages 105-144 (1993), which is herein incorporated by reference. In another embodiment, one or more binders are combined with one or more other non-limiting examples of alumina materials such as aluminum oxyhydroxide, γ-alumina, boehmite, diaspore, and transitional aluminas such as α-alumina, β-alumina, γ-alumina, δ-alumina, ε-alumina, κ-alumina, and σ-alumina, aluminum trihydroxide, such as gibbsite, bayerite, nordstrandite, doyelite, and mixtures thereof.

In another embodiment, the binders are alumina sols, predominantly comprising aluminum oxide, optionally including some silicon. In yet another embodiment, the binders are peptized alumina made by treating alumina hydrates such as pseudobohemite, with an acid, preferably an acid that does not contain a halogen, to prepare sols or aluminum ion solutions. Non-limiting examples of commercially available colloidal alumina sols include Nalco 8676 available from Nalco Chemical Co., Naperville, Ill., and Nyacol available from The PQ Corporation, Valley Forge, Pa.

The molecular sieve, in a preferred embodiment, is combined with one or more matrix materials. Matrix materials are typically effective in reducing overall catalyst cost, act as thermal sinks assisting in shielding heat from the catalyst composition for example during regeneration, densifying the catalyst composition, increasing catalyst strength such as crush strength and attrition resistance, and to control the rate of conversion in a particular process.

Non-limiting examples of matrix materials include one or more of: rare earth metals, metal oxides including titania, zirconia, magnesia, thoria, beryllia, quartz, silica or sols, and mixtures thereof, for example silica-magnesia, silica-zirconia, silica-titania, silica-alumina and silica-alumina-thoria. In an embodiment, matrix materials are natural clays such as those from the families of montmorillonite and kaolin. These natural clays include sabbentonites and those kaolins known as, for example, Dixie, McNamee, Georgia and Florida clays. Non-limiting examples of other matrix materials include: haloysite, kaolinite, dickite, nacrite, or anauxite. In one embodiment, the matrix material, preferably any of the clays, are subjected to well known modification processes such as calcination and/or acid treatment and/or chemical treatment.

In one preferred embodiment, the matrix material is a clay or a clay-type composition, preferably the clay or clay-type composition having a low iron or titania content, and most preferably the matrix material is kaolin. Kaolin has been found to form a pumpable, high solid content slurry, it has a low fresh surface area, and it packs together easily due to its platelet structure. A preferred average particle size of the matrix material, most preferably kaolin, is from about 0.1 μm to about 0.6 μm with a D90 particle size distribution of less than about 1 μm.

In another embodiment, the weight ratio of the binder to the matrix material used in the formation of the molecular sieve catalyst composition is from 0:1 to 1:15, preferably 1:15 to 1:5, more preferably 1:10 to 1:4, and most preferably 1:6 to 1:5. It has been found that a higher sieve content, lower matrix content, increases the molecular sieve catalyst composition performance, however, lower sieve content, higher matrix material, improves the attrition resistance of the composition.

In another embodiment, the formulated molecular sieve catalyst composition contains from about 1% to about 99%, more preferably from about 5% to about 90%, and most preferably from about 10% to about 80%, by weight of the molecular sieve based on the total weight of the molecular sieve catalyst composition.

In another embodiment, the weight percent of binder in or on the spray dried molecular sieve catalyst composition based on the total weight of the binder, molecular sieve, and matrix material is from about 2% by weight to about 30% by weight, preferably from about 5% by weight to about 20% by weight, and more preferably from about 7% by weight to about 15% by weight.

Once the molecular sieve catalyst composition is formed in a substantially dry or dried state, to further harden and/or activate the formed catalyst composition, a heat treatment such as calcination, at an elevated temperature is usually performed. A conventional calcination environment is air that typically includes a small amount of water vapor. Typical calcination temperatures are in the range from about 400° C. to about 1,000° C., preferably from about 500° C. to about 800° C., and most preferably from about 550° C. to about 700° C., preferably in a calcination environment such as air, nitrogen, helium, flue gas (combustion product lean in oxygen), or any combination thereof.

D. General Conditions for Converting Methanol to Olefins

According to the reaction process of this invention, oxygenate is contacted with olefin forming catalyst to form an olefin product, particularly ethylene and propylene. The process for converting the oxygenate feedstock is, preferably, a continuous fluidized bed process, and most preferably a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977, which are all herein fully incorporated by reference.

One preferred reactor type is a riser reactor. These types of reactors are generally described in Riser Reactor, Fluidization and Fluid-Particle Systems, pages 48 to 59, F. A. Zenz and D. F. Othmo, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In one embodiment of the invention, a fluidized bed process or high velocity fluidized bed process includes a reactor system, catalyst separation system, and a regeneration system. The reactor system preferably is a fluid bed reactor system. In one embodiment, the fluid bed reactor system has a first reaction zone within one or more riser reactors, and a second reaction zone within at least one catalyst separation vessel, preferably comprising one or more cyclones. In one embodiment, one or more riser reactors and catalyst separation vessel is contained within a single reactor vessel.

An oxygenate stream, preferably containing one or more oxygenates, and optionally one or more diluents, is fed to a fluid bed reactor in which a molecular sieve catalyst composition is introduced. In one embodiment, the molecular sieve catalyst composition is contacted with a liquid or gas, or combination thereof, prior to being introduced to the riser reactor. Preferably, the liquid is water or methanol, and the gas is an inert gas such as nitrogen.

The oxygenate in the oxygenate feed stream entering the reactor system is preferably converted, partially or fully, in a reactor zone forming an olefin product and a coked catalyst. The olefin product and coked catalyst, as well as any unconverted or unreacted oxygenate, are sent to a catalyst separation vessel where the coked catalyst is separated from the olefin product and the unconverted or unreacted oxygenate.

In a preferred embodiment, cyclones within the separation vessel are used to separate the coked catalyst composition. Gravity effects within the disengaging vessel can also be effective in separating the catalyst. Other processs for separating the catalyst from the gaseous effluent include the use of plates, caps, elbows, and the like.

The average reaction temperature employed in the conversion process, specifically within the reactor, is from about 250° C. to about 800° C. Preferably the average reaction temperature within the reactor is from about 250° C. to about 750° C.; more preferably, from about 300° C. to about 650° C.; yet more preferably from about 350° C. to about 600° C.; and most preferably from about 400° C. to about 500° C.

The pressure employed in the conversion process, specifically within the reactor, is not critical. The reaction pressure is based on the partial pressure of the feedstock exclusive of any diluent therein. Typically the reaction pressure employed in the process is in the range of from about 0.1 kPa abs. to about 5 MPa abs., preferably from about 5 kPa abs. to about 1 MPa abs., and most preferably from about 20 kPa abs. to about 500 kPa abs.

The weight hourly space velocity (WHSV), particularly in a process for converting a feedstock containing one or more oxygenates in the presence of a molecular sieve catalyst composition within a reaction zone, is defined as the total weight of the feedstock excluding any diluents to the reaction zone per hour per weight of molecular sieve in the molecular sieve catalyst composition in the reaction zone. The WHSV is maintained at a level sufficient to keep the catalyst composition in a fluidized state within a reactor.

Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing methanol and dimethyl ether is in the range of from about 20 hr⁻¹ to about 300 hr⁻¹.

The superficial gas velocity (SGV) of the feedstock including diluent and reaction products within the reactor is preferably sufficient to fluidize the molecular sieve catalyst composition within a reaction zone of the reactor. The SGV in the process, particularly within the reactor system, more particularly within a riser reactor, is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec.

According to one embodiment, the conversion of oxygenate, particularly the conversion of methanol, is from 90 wt % to 98 wt %. According to another embodiment the conversion of methanol is from 92 wt % to 98 wt %, preferably from 94 wt % to 98 wt %.

According to another embodiment, the conversion of methanol is above 98 wt % to less than 100 wt %. According to another embodiment, the conversion of methanol is from 98.1 wt % to less than 100 wt %; preferably from 98.2 wt % to 99.8 wt %. According to another embodiment, the conversion of methanol is from 98.2 wt % to less than 99.5 wt %; preferably from 98.2 wt % to 99 wt %.

It is desirable to maintain an amount of coke on the catalyst in the reaction vessel to enhance the formation of desired olefin product, particularly ethylene and propylene. It is particularly desirable that the catalyst in the reactor be maintained to contain at least about 1.5 wt % coke. Preferably, the amount of coke maintained on the catalyst in the reactor should be from about 2 wt % to about 30 wt %.

The production of olefins forms a substantial amount of water as a by-product. Much of this water by-product can be removed prior to distillation by cooling the olefin stream to a temperature below the condensation temperature of the water vapor in the stream. Preferably, the temperature of the olefin stream is cooled to a temperature below the condensation temperature of the oxygenate stream. In certain embodiments it is desirable to cool the olefin stream below the condensation temperature of methanol.

It is desirable to cool the olefin stream from the oxygenate to olefin reaction process, then separate the cooled olefin stream into a condensed, water-containing stream and an olefin vapor stream. The condensed, water-containing stream comprises most of the water from the olefin stream, and a significant portion of the undesirable oxygenated hydrocarbons from the olefin stream. The olefin vapor stream comprises a majority of the desired olefins, e.g., ethylene and propylene. This olefin vapor stream will be in condition to send to a compressor system for compression and separation into olefin component streams. Such a stream will have the acceptable diene content so that compressor fouling can be minimized.

A quench column is one type of equipment that is effective in cooling the olefin stream. In a quench column, a quenching fluid is directly contacted with the olefin stream to cool the stream to the desired condensation temperature. Condensation produces the condensed water-containing stream, which is also referred to as a heavy bottoms stream. The olefin portion of the olefin product stream remains a vapor, and exits the quench column as an overhead vapor stream. The overhead vapor stream is rich in olefin product, and can also contain some oxygenated hydrocarbon by-products as well as water.

In one embodiment, the quenching fluid is a recycle stream of the condensed water-containing, heavy bottoms stream of the quench column. This water-containing recycle stream is desirably cooled, e.g., by a heat exchanger, and injected back into the quench column. It is preferred in this embodiment to not inject cooling medium from an outside source into the quench column, although it may be desirable to do so in other separation equipment down stream of the quench column.

V. Separation of Light Olefin from By-Products

In one embodiment of the invention, light olefins, particularly ethylene and propylene, are separated from heavier by-products such as hydrocarbons having a boiling point greater than that of propylene, including propane, C₄+ olefins, C₅+ olefins, or higher hydrocarbons, which are produced in the carbon oxide conversion zone. C₄+ olefins as defined herein refer to olefin compounds, as well as other hydrocarbons, having a boiling point greater than that of propane. C₅+ olefins as defined herein refer to olefin compounds, as well as other hydrocarbons, having a boiling point greater than that of butane.

Removal of by-products such as hydrocarbons having a boiling point greater than that of propylene, hydrocarbons having a boiling point less than that of ethylene, or other undesirable products from an olefin stream to separate out the light olefins can be accomplished by any conventional means capable of removing such by-product to the appropriate level. Conventional distillation techniques are particularly desirable methods, and temperatures and pressures effective for separating by-products from ethylene and/or propylene according to boiling point are used.

In order to obtain a particularly high degree of separation between the by-products from ethylene and/or propylene, extractive distillation is optionally used. Extractive distillation differs from conventional distillation to the extent that the ethylene and/or propylene is distilled from the by-products using an extractant to aid the distillation process.

In one embodiment, extractive distillation is carried out using a vessel or tower (an oxygenate removal tower) having internal packing or trays that create a temperature difference from top to bottom of the tower. The upper portion of the oxygenate removal tower is the cooler portion, and it is in this portion of the tower that higher volatile components in the feed exit as an effluent stream. Preferably the effluent stream includes ethylene, propylene, and optionally butylenes. More preferably, the effluent stream includes ethylene and propylene.

An extractant or solvent is added to an extractive distillation vessel or tower so as to enhance or suppress the volatility of oxygenate contaminants, e.g., the undesired by-products, relative to the olefin. This makes it easier to distill the oxygenates from the olefin, resulting in removal of the oxygenates to very low levels.

Extractants which can be used in this invention are compositions that exist in the liquid phase at a pressure of about 1 atm. These extractants also desirably have an average boiling point of at least about 38° C., preferably at least about 49° C., and more preferably at least about 66° C., at 1 atm. Average boiling point, as defined herein, takes into account the boiling point of each compound in the extractant on a weight average basis. For example, an extractant containing 90 wt % of a compound having a boiling point of 100° C., and 10 wt % of a compound having a boiling point of 200° C., both at 1 atm, would have an average boiling point of 110° C., at 1 atm.

The extractants are desirably polar compositions. Such compositions preferably contain compounds such as water, monohydric alcohols, polyhydric alcohols, or mixtures thereof. Preferred monohydric alcohols include methanol, ethanol and propanol. Preferred polyhydric alcohols include glycols. Preferred glycols include ethylene glycol and tri-ethylene glycol. It is desirable that the extractant contain at least about 75 wt % water, monohydric alcohol, and/or polyhydric alcohol, preferably at least about 85 wt %, more preferably at least about 90 wt %, and most preferably at least about 95 wt %. Methanol is most preferred as the extractant in this invention.

The extractive distillation process is particularly effective for removing very low concentrations of oxygenate contaminants present in the olefin vapor stream. In one embodiment of the invention, extractant is added at an upper region of an extractive distillation column, and the olefin vapor stream is added at a side region of the column. Separated ethylene and/or propylene is recovered from the column in a first fraction, preferably from an upper portion of the column, and the separated by-products are recovered in a second fraction, preferably from a lower portion of the column. A majority of the added extractant (i.e., at least 50% of the amount added to the column) is also recovered from a lower portion of the column, preferably along with the recovered by-products.

In one embodiment of the invention, the separated by-product stream comprises at least a majority (i.e., at least 50%) of the by-products present in the provided olefin vapor stream. Preferably, this stream comprises at least about 5 wt % C₄+ olefins, more preferably about 10 wt % C₄+ olefins, most preferably about 15 wt % C₄+ olefins, based on the total weight of the stream. The by-product stream optionally includes acetaldehyde and/or dimethyl ether. In one embodiment, the by-product stream comprises at least about 1,000 wppm acetaldehyde and/or dimethyl ether; in another, at least about 1,500 wppm; and in yet another, at least about 2,000 wppm, based on the total weight of the stream.

In yet another embodiment, a light olefin stream is separated and recovered, with the recovered light olefin stream containing at least 50 wt % of at least one C₂ to C₄ olefin, based on total composition of the light olefin stream. Preferably the light olefins stream contains at least 60 wt % of at least one C₂ to C₄ olefin, more preferably at least 70 wt % of at least one C₂ to C₄ olefin, and most preferably at least 80 wt % of at least one C₂ to C₄ olefin, based on total weight of the light olefin stream.

In another embodiment, the light olefin stream is further separated into components streams. These components streams include at least one additional by-product stream. The additional by-product stream preferably contains one or more hydrocarbons having a boiling point greater than that of propylene. Alternatively, the additional by-product stream contains one or more hydrocarbons having a boiling point less than that of ethylene. The by-products can be further processed or sent to a synthesis gas production zone to produce a synthesis gas. The synthesis gas is desirably used for feed in a carbon oxide conversion zone.

VI. Recovery and Conversion of By-Products to Light Olefins

This invention includes recovery and conversion of at least one by-product stream to form additional light olefins. Such by-products include a substantial amount of hydrocarbons. Desirably, the by-product stream that is recovered and converted to form additional light olefins includes at least 30 wt % hydrocarbons having a boiling point greater than that of propylene, based on total weight of the by-product stream. Preferably, the by-product stream includes at least 50 wt %, more preferably at least 70 wt %, and most preferably at least 80 wt % hydrocarbons, which have a boiling point greater than that of propylene, based on total composition of the by-product stream.

Alternatively, the by-product stream that is recovered and converted to form additional light olefins includes at least 30 wt % hydrocarbons having a boiling point less than that of ethylene, based on total weight of the by-product stream. Preferably, the by-product stream includes at least 50 wt %, more preferably at least 70 wt %, and most preferably at least 80 wt % hydrocarbons, which have a boiling point less than that of ethylene, based on total composition of the by-product stream.

In one embodiment of the invention, a by-product stream is separated and recovered from the olefin product stream produced in the oxygenate conversion zone. The recovered by-product stream is then converted to a synthesis gas stream, and the synthesis gas stream is sent to a carbon oxide conversion zone to convert the carbon oxides in the synthesis gas to a methanol stream. The methanol stream is then sent to an oxygenate conversion zone for conversion of the methanol and other oxygenates in the methanol stream to additional olefins.

The recovered by-product stream is converted to synthesis gas using any conventional type of synthesis gas plant as described above. Preferably, the synthesis gas plant is a steam reforming process, a partial oxidation process, or a combination of the two. More preferably, the synthesis gas plant is a partial oxidation process.

The synthesis gas plant, which uses the by-product stream as a feed stream to form synthesis gas, can be the same as the system described in the integrated system above or it can be a separate or different system. In one embodiment, the synthesis gas plant, which is used to convert the by-product stream to synthesis gas is a separate system, preferably a separate partial oxidation system.

The synthesis gas stream produced from the by-product stream is optionally treated to remove impurities and/or poisons prior to entering the carbon oxide conversion zone. Streams containing these impurities can also be passed to a synthesis gas production zone to produce a synthesis gas stream. Optionally, a purge stream can be used to purge off some of a portion of the synthesis gas stream so as to reduce certain impurities.

VII. Acid Gas Treating the Olefin Stream

In one embodiment of the invention, the ethylene and/or propylene stream that is separated from the olefin product stream from the oxygenate conversion zone is treated to remove entrained acid gases such as CO₂ that may also be present in this stream. Solid or liquid acid gas treatment systems can be used. In either system, the acid gas is removed from the ethylene and/or propylene stream by contacting the ethylene and/or propylene stream with an acid gas absorbent or adsorbent. Examples of such absorbents or adsorbents include amines, potassium carbonate, caustic, alumina, molecular sieves, and membranes, particularly membranes formed of polysulfone, polyimid, polyamide, glassy polymer and cellulose acetate. Solutions containing amines and caustic compounds are preferred, with caustic compounds being more preferred.

Aqueous amine solutions which are useful in this invention can contain any amine compound or compounds suitable for acid gas absorption. Examples include alkanolamines, such as triethanolamine (TEA); methyldiethanolamine (MDEA); diethanolamine (DEA); monoethanolamine (MEA); diisopropanolamine (DIPA); and hydroxyaminoethyl ether (DGA). Effective concentrations can range from about 0.5 to about 8 moles of amine per liter of aqueous solution.

Piperazine and/or monomethylethanolamine (MMEA) can be added to aqueous amine solutions to enhance their absorption capabilities. These additives can be included in the aqueous solution at a concentration of from about 0.04 to about 2 moles per liter of aqueous solution.

Caustic compounds which can be used in this invention are alkaline compounds which are effective in removing acid gas from an olefin stream. Examples of such alkaline compounds include sodium hydroxide and potassium hydroxide.

VIII. Washing and Drying the Olefin Stream

This invention further includes an optional drying embodiment. In this embodiment, a solid or liquid drying system can be used to remove water and/or additional oxygenated hydrocarbons from the ethylene and/or propylene stream. In a preferred embodiment, the ethylene and/or propylene is dried following acid gas treating.

In the solid drying system, the ethylene and/or propylene stream is contacted with a solid adsorbent to further remove water and oxygenated hydrocarbon to very low levels. Typically, the adsorption process is carried out in one or more fixed beds containing a suitable solid adsorbent.

Adsorption is particularly useful for removing water and oxygenated hydrocarbons to very low concentrations, and for removing oxygenated hydrocarbons that may not normally be removed by using other treatment systems. Preferably, an adsorbent system used as part of this invention has multiple adsorbent beds. Multiple beds allow for continuous separation without the need for shutting down the process to regenerate the solid adsorbent. For example, in a three bed system typically one bed is on-line, one bed is regenerated off-line, and a third bed is on stand-by.

The specific adsorbent solid or solids used in the adsorbent beds depends on the types of contaminants being removed. Examples of solid adsorbents for removing water and various polar organic compounds, such as oxygenated hydrocarbons and absorbent liquids, include aluminas, silica, 3 angstroms molecular sieves, 4 angstroms molecular sieves, and alumino-silicates. Beds containing mixtures of these sieves or multiple beds having different adsorbent solids can be used to remove water, as well as a variety of oxygenated hydrocarbons.

In this invention, one or more adsorption beds can be arranged in series or parallel. In one example of a series arrangement, a first bed is used to remove the smallest and most polar molecules which are the easiest to remove. Subsequent beds for removing larger less polar oxygenated species are next in series. As a specific example of one type of arrangement, water is first selectively removed using a 3 angstroms molecular sieve. This bed is then followed by one or more beds containing one or more less selective adsorbents such as a larger pore molecular sieve e.g. 13× and/or a high surface area active alumina such as Selexorb CD (Alcoa tradename).

In another embodiment, the first bed is a 3.6 angstroms molecular sieve capable of selectively removing both water and methanol. This bed can then be followed by one or more 13× or active alumina beds as described above.

The adsorbent beds can be operated at ambient temperature or at elevated temperature as required, and with either upward or downward flow. Regeneration of the adsorbent materials can be carried out by conventional processes including treatment with a stream of a dry inert gas such as nitrogen at elevated temperature.

In the liquid drying system, a water absorbent is used to remove water from the compressed ethylene and/or propylene stream. The water absorbent can be any liquid effective in removing water from an olefin stream. Examples of water absorbents include alcohols, amines, amides, nitriles, heterocyclic nitrogen containing compounds, or a combination of any of the preceding. Either monohydric alcohols or polyhydric alcohols can be used as the alcohol absorbent. Specific examples of absorbents include methanol, ethanol, propanol, ethylene glycol, diethylene glycol, triethylene glycol, ethanolamine, diethanolamine, triethanolamine, hindered cyclic amines, acetonitrile, n-methylpyrrolidone, dimethyl formamide, and combinations thereof.

To obtain a substantial degree of effectiveness, the water absorbent should contain little non-water absorbing components. For example, the water absorbent should contain at least about 75 wt % water absorbing components. Desirably, the water absorbent contains at least about 90 wt %, preferably at least about 95 wt %, and most preferably at least about 98 wt % water absorbent.

IX. Ethylene, Propylene Recovery and Derivative Processes

The olefin stream produced in the oxygenate conversion zone is desirably separated into olefin components so that high purity ethylene and/or propylene can be recovered. According to this invention, high purity is defined as at least about 95 wt %, based on total weight of the stream. Preferably, the ethylene and/or propylene stream comprises at least about 98 wt % ethylene and/or propylene, and most preferably at least about 99 wt % ethylene and/or propylene, based on total weight of the ethylene and/or propylene stream.

The ethylene and/or propylene stream separated according to this invention can be polymerized in a polymerization zone to form plastic compositions, e.g., polyolefins, particularly polyethylene and polypropylene. Any conventional process for forming polyethylene or polypropylene can be used. Catalytic processes are preferred. Particularly preferred are metallocene, Ziegler/Natta, aluminum oxide and acid catalytic systems. See, for example, U.S. Pat. Nos. 3,258,455; 3,305,538; 3,364,190; 5,892,079; 4,659,685; 4,076,698; 3,645,992; 4,302,565; and 4,243,691, the catalyst and process descriptions of each being expressly incorporated herein by reference. In general, these processes involve contacting the ethylene and/or propylene stream with a polyolefin-forming catalyst at a pressure and temperature effective to form the polyolefin product.

In one embodiment of this invention, the ethylene and/or propylene stream is contacted with a metallocene catalyst to form a polyolefin. Desirably, the polyolefin forming process is carried out at a temperature ranging between about 50° C. and about 320° C. The reaction can be carried out at low, medium or high pressure, being anywhere within the range of about 1 bar to about 3200 bar. For processes carried out in solution, an inert diluent can be used. In this type of operation, it is desirable that the pressure be at a range of from about 10 bar to about 150 bar, and preferably at a temperature range of from about 120° C. to about 250° C. For gas phase processes, it is preferred that the temperature generally be within a range of about 60° C. to about 120° C., and that the operating pressure be from about 5 bar to about 50 bar.

In addition to polyolefins, numerous other olefin derivatives may be formed from the ethylene and/or propylene stream separated according to this invention. The olefins separated according to this invention can also be used in the manufacture of such compounds as aldehydes, acids such as C₂-C₁₃ mono carboxylic acids, alcohols such as C₂-C₁₂ mono alcohols, esters made from the C₂-C₁₂ mono carboxylic acids and the C₂-C₁₂ mono alcohols, linear alpha olefins, vinyl acetate, ethylene dicholoride and vinyl chloride, ethylbenzene, ethylene oxide, cumene, acrolein, allyl chloride, propylene oxide, acrylic acid, ethylene-propylene rubbers, and acrylonitrile, and trimers and dimers of ethylene and propylene.

Having now fully described this invention, it will be appreciated by those skilled in the art that the invention can be performed within a wide range of parameters within what is claimed, without departing from the spirit and scope of the invention.

1. A process for the production of light olefins from a hydrocarbon feed stream, the process comprising the steps of:
a) providing a hydrocarbon feed stream;
b) passing the hydrocarbon feed stream to a synthesis gas production zone to produce a synthesis gas stream;
c) passing the synthesis gas stream to a carbon oxide conversion zone to produce a methanol stream;
d) passing the methanol stream to an oxygenate conversion zone to produce an olefin stream;
e) separating the olefin stream into component streams, one component stream comprised of a light olefin stream and another component stream comprised of a by-product stream; and
f) passing the by-product stream to a synthesis gas production zone to produce a synthesis gas stream.

2. The process of claim 1, wherein the light olefin stream is comprised of at least 50 wt % of at least one C₂ to C₄ olefin, based on total weight of the light olefin stream.

3. The process of claim 1, wherein the by-product stream is comprised of at least 30 wt % hydrocarbons having a boiling point greater than that of propylene, based on total weight of the by-product stream.

4. The process of claim 1, further comprising the step of: g) passing the synthesis gas stream produced in step f) to a carbon oxide conversion zone to produce a methanol stream.

5. The process of claim 1, wherein the hydrocarbon feed stream includes methane.

6. The process of claim 1, wherein the synthesis gas production zone of either step b) or step f) is a partial oxidation process.

7. The process of claim 1, wherein the synthesis gas production zone of either step b) or step f) is a reforming process.

8. The process of claim 1, wherein the synthesis gas production zone includes a catalyst.

9. The process of claim 8, wherein the catalyst is a nickel containing catalyst.

10. The process of claim 1, wherein the synthesis gas stream in step step b) and in step f) has a molar ratio of hydrogen to carbon oxide of from 1:1 to 5:1.

11. The process of claim 4, wherein the carbon oxide conversion zone in step g) is different from the carbon oxide conversion zone in step c).

12. The process of claim 4, wherein the carbon oxide conversion zone is step g) is the same as the carbon oxide conversion zone in step c).

13. The process of claim 4, wherein the carbon oxide conversion zone of either step c) or step g) includes a catalyst comprising at least one transition element selected from the group consisting of Ni, Co, Pd, Ru, Rh, Ir, Pt, Os and Fe.

14. The process of claim 1, wherein the oxygenate conversion zone includes a catalyst containing silicoaluminophosphate molecular sieve.

15. The process of claim 1 1, wherein the silicoaluminophosphate molecular sieve is selected from the group consisting of SAPO-18, SAPO-34, SAPO-35, SAPO-44, SAPO-47, and a mixture thereof.

16. The process of claim 1, wherein the olefin stream is separated into component streams by distillation.

17. The process of claim 1, wherein 90 wt % to 98 wt % of the methanol stream that is passed to the oxygenate conversion zone in step d) is converted to produce the olefin stream.

18. The process of claim 1, wherein 98 wt % to less than 100 wt % of the methanol stream that is passed to the oxygenate conversion zone in step d) is converted to produce the olefin stream.

19. The process of claim 1, further comprising passing the light olefin stream of step e) to a polymerization zone to produce a polyolefin stream.

20. The process of claim 1, wherein the light olefin stream in step e) is further separated into component streams, including an additional by-product stream, and the additional by-product stream is passed to a synthesis gas production zone to produce a synthesis gas stream.

21. A process for the production of light olefins from a hydrocarbon feed stream, the process comprising the steps of:
a) providing a C, to C₅ hydrocarbon containing feed stream;
b) passing the hydrocarbon feed stream to a synthesis gas production zone to produce a synthesis gas stream;
c) passing the synthesis gas stream to a carbon oxide conversion zone to produce a methanol stream;
d) passing the methanol stream to an oxygenate conversion zone to produce an olefin stream;
e) separating the olefin stream into component streams, one component stream comprised of a light olefin stream and another component stream comprised of a by-product stream; and
f) passing the by-product stream to a synthesis gas production zone to produce a synthesis gas stream.

22. The process of claim 21, wherein the light olefin stream is comprised of at least 50 wt % of at least one C₂ to C₄ olefin, based on total weight of the light olefin stream.

23. The process of claim 21, wherein the by-product stream is comprised of at least 30 wt % hydrocarbons having a boiling point greater than that of propylene, based on total weight of the by-product stream.

24. The process of claim 21, further comprising the step of:
g) passing the synthesis gas stream produced in step f) to a carbon oxide conversion zone to produce a methanol stream.

25 The process of claim 24, further comprising the step of:
h) passing the methanol stream produced in step g) to an oxygenate conversion zone to produce an olefin stream.

26. The process of claim 21, wherein the hydrocarbon feed stream is a gas stream comprising at least 50% methane by volume, based on total volume of the feed stream.

27. The process of claim 21, wherein the synthesis gas production zone of either step b) or step f) is a partial oxidation process.

28. The process of claim 21, wherein the synthesis gas production zone of either step b) or step f) is a reforming process.

29. The process of claim 21, wherein the synthesis gas production zone includes a catalyst.

30. The process of claim 29, wherein the catalyst is a nickel containing catalyst.

31. The process of claim 21, wherein the synthesis gas stream in step step b) and in step f) has a molar ratio of hydrogen to carbon oxide of from 1:1 to 5:1.

32. The process of claim 24, wherein the carbon oxide conversion zone in step g) is different from the carbon oxide conversion zone in step c).

33. The process of claim 24, wherein the carbon oxide conversion zone is step g) is the same as the carbon oxide conversion zone in step c).

34. The process of claim 24, wherein the carbon oxide conversion zone of either step c) or step g) includes a catalyst comprising at least one transition element selected from the group consisting of Ni, Co, Pd, Ru, Rh, Ir, Pt, Os and Fe.

35. The process of claim 21, wherein the oxygenate conversion zone includes a catalyst containing silicoaluminophosphate molecular sieve.

36. The process of claim 35, wherein the silicoaluminophosphate molecular sieve is selected from the group consisting of SAPO-18, SAPO-34, SAPO-35, SAPO-44, SAPO-47, and a mixture thereof.

37. The process of claim 21, wherein the olefin stream is separated into component streams by distillation.

38. The process of claim 21, wherein 90 wt % to 98 wt % of the methanol stream that is passed to the oxygenate conversion zone in step d) is converted to produce the olefin stream.

39. The process of claim 21, wherein 98 wt % to less than 100 wt % of the methanol stream that is passed to the oxygenate conversion zone in step d) is converted to produce the olefin stream.

40. The process of claim 21, further comprising passing the light olefin stream of step e) to a polymerization zone to produce a polyolefin stream.

41. The process of claim 21, wherein the light olefin stream in step e) is further separated into component streams, including an additional by-product stream, and the additional by-product stream is passed to a synthesis gas production zone to produce a synthesis gas stream.

42. A process for producing olefins, the process comprising the steps of:
a) separating an olefin stream into component streams, one component stream comprised of a light olefin stream and another component stream comprised of a by-product stream, the light olefin stream comprised of at least 50 wt % of at least one C₂ to C₄ olefin, based on total weight of the light olefin stream, and the by-product stream comprised of at least 30 wt % hydrocarbons having a boiling point greater than that of propylene, based on total weight of the by-product stream; and
b) passing the by-product stream to a synthesis gas production zone to produce a synthesis gas stream.

43. The process of claim 42, wherein the synthesis gas production zone is a partial oxidation process.

44. The process of claim 42, wherein the synthesis gas production zone is a reforming process.

45. The process of claim 42, wherein the synthesis gas production zone includes a catalyst.

46. The process of claim 45, wherein the catalyst is a nickel containing catalyst.

47. The process of claim 42, wherein the synthesis gas stream has a molar ratio of hydrogen to carbon oxide of from 1:1 to 5:1.

48. The process of claim 42, further comprising the step of:
c) passing the synthesis gas stream to a carbon oxide conversion zone to produce a methanol stream.

49. The process of claim 48, wherein the carbon oxide conversion zone includes a catalyst comprising at least one transition element selected from the group consisting of Ni, Co, Pd, Ru, Rh, Ir, Pt, Os and Fe.

50. The process of claim 49, further comprising the step of:
d) passing the methanol stream to an oxygenate conversion zone to produce an olefin stream.

51. The process of claim 50, wherein the oxygenate conversion zone includes a catalyst containing silicoaluminophosphate molecular sieve.

52. The process of claim 51, wherein the silicoaluminophosphate molecular sieve is selected from the group consisting of SAPO-18, SAPO-34, SAPO-35, SAPO-44, SAPO-47, and a mixture thereof.

53. The process of claim 42, wherein the olefin stream is separated into component streams by distillation.

54. The process of claim 50, wherein 90 wt % to 98 wt % of the methanol stream that is passed to the oxygenate conversion zone is converted to produce the olefin stream.

55. The process of claim 50, wherein 98 wt % to less than 100 wt % of the methanol stream that is passed to the oxygenate conversion zone is converted to produce the olefin stream.

56. The process of claim 42, further comprising passing the light olefin stream to a polymerization zone to produce a polyolefin stream.
